(19) United States
(12) Patent Application Publication
(10) Pub. No.: US 2006/0116774 A1
(43) Pub. Date: Jun. 1, 2006

(54) Prosthetic acetabular cup and method of manufacture

(75) Inventors: Eric Jones, Limerick (IE); Patrick Raugel, Ramsey, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 11/245,645

(22) Filed: Oct. 7, 2005

(30) Foreign Application Priority Data
Oct. 12, 2004 (GB) 0422666.8

(51) Int. Cl. A61F 2/34

(52) U.S. Cl. 623/22.32; 427/2.27

(57) ABSTRACT

A prosthetic bearing element and a method for forming the same includes an injection molded bearing made of PEEK resin with short carbon fiber reinforcement. The inner surface of the PEEK bearing is adapted to receive a ceramic or metal articulation component. The outer surface of the bearing layer includes sputtered titanium particles forming a porous backing layer. Hydroxyapatite is then sputtered or otherwise deposited onto the titanium backing layer to form an outer surface of the prosthetic bearing element. A barrier layer can be formed either of PEEK or titanium which layer is between the outer surface of the molded bearing and the inner surface of the porous structure. The barrier layer prevents tissue ingrowth into the bearing component. Hydroxyapatite is then sputtered onto the outer porous layer or applied by solution deposition. This outer surface of the prosthetic bearing element can then be coated with bone morphogenic protein.

### BACKGROUND OF THE INVENTION

This invention relates to prosthetic acetabular cups and to methods of making them.

The invention is intended to improve the long term attachment to bone of an implant that incorporates the benefits of composite materials for structural and bearing functions.

Prosthetic metallic acetabular cup implants and assemblies are usually much stiffer than the surrounding bone and this stiffness of the acetabular cup causes changes of density in the bony structure surrounding the cup. U.S. Pat. No. 5,609,646 relates to an elastic acetabular cup which has now demonstrated in vivo its efficacy.

The applicants have developed a composite material made of polyetheretherketone (PEEK) resin and 20 to 40% of short carbon fibers, preferably with 30% short carbon fibers. This material has demonstrated good wear resistance properties and a prosthetic bearing component comprising these materials is described in U.S. Pat. No. 6,638,311.

Hydroxyapatite (HAP) coating activates bone cells attachment but HAP resorbs and bone cells come directly in contact with the material of the implant (which is usually made of a titanium alloy or of the composite material described in U.S. Pat. No. 6,638,311). Some material is more prone to encourage bone cell adherence and development. Beneath the hydroxyapatite layer, the surface roughness, porosity and purity do have an effect on the bone cells development. Pure titanium with a roughness in a range of 4 to 7 μm Ra, 30 to 40 μm Rz, and 35 to 65 μm Rt is known for encouraging bone cells adherence and growth so creating a microlock between newly formed bone and the implant.

Open pores at the surface of the implant are rooms for bone trabeculae formation and deep interdigitation. This mechanical interlocking is able to provide long term attachment after complete dissolution of the hydroxyapatite coating. Composite/plastic materials are not X-ray lucent.

According to the present invention a prosthetic acetabular cup includes a bearing surface layer made from a composite material including PEEK resin and at least 20 to 40% short carbon fibers, and a backing layer or layers to provide a barrier and/or porosity and/or roughness. The backing layer or layers can be coated with a bioactive material if required.

Depending on the coating properties one or more of the following three aspects of the invention is fulfilled:
create a barrier between the composite materials and the bone cells;
provide an appropriate roughness for bone cell attachment;
provide open porosity for bone cells ingrowth.

The backing layer can be made from metal, for example titanium, titanium alloy, cobalt chrome alloy, tantalum or niobium, or from, for example, pure PEEK to produce a barrier between the composite material and the bone cells.

These and other aspects of the invention are provided by a prosthetic acetabular cup which has a bearing surface layer made from a composite material such as, for example, PEEK resin having at least 20%-40% short carbon fibers and a backing layer or layers providing a barrier and/or porosity and/or roughness. Preferably the backing layer is made from metal and is coated with a bioactive material. The backing layer could also be made from PEEK resin to produce a barrier between the composite material or the surface layer and the bone cells in which it will be used. The acetabular cup outer layer may be made of a bioactive material such as hydroxyapatite with or without bone morphogenic proteins.

The method of forming the prosthetic bearing surface has as a first step injection molding and bearing layer of PEEK resin having 20%-40% carbon fiber to form an inner bearing surface. Then metal particles are sputtered on to the outer nonbearing surface of the molded bearing to form a porous backing layer and then hydroxyapatite is sputtered on to the metal backing layer to form an outer surface of the prosthetic bearing element. PEEK may be plasma sprayed on the outer surface of the bearing layer prior to applying the metal layer. The metal particle size may be varied to form an interconnected porosity which increases towards the outer surface as the layer is built up. The particle size may increase from smaller to larger to form the increasing interconnected porosity. A mixture of metal particles and hydroxyapatite particles may be sputtered on to the backing layer to form the interconnected porosity. In a most preferred embodiment the bearing layer has about 30% short carbon fibers. Also preferably there is a metal layer between the PEEK bearing and the porous metal layer for blocking tissue ingrowth beyond the porous metal layer. The bearing layer may be preformed and the backing layer or layers may be applied by sputtering and/or chemical or plasma deposition. The metal utilized for the backing layer or layers is preferably selected from the group consisting of titanium, titanium alloy, tantalum, niobium or cobalt chrome alloy.

The benefit of the construction is that bioactive material encourages the bone cells apposition and development; rough surface and/or porous surface provides structure for mechanical fixation after dissolution of the bioactive layer; composite material provides elasticity for natural load distribution to the bone, and also provides highly wear resistant bearing surface; and the benefits of the metallic material, when provided, is to provide an opaque marker for X-rays and a proven biological interface for good bone ongrowth/ingrowth. The bioactive material can be hydroxyapatite (HAP) and/or bone morphogenic proteins (BMP).

The invention also includes a method of making an acetabular cup which includes a bearing surface layer made from a composite material including PEEK resin and at least 20 to 40% short carbon fibers, a backing layer or layers to provide a barrier and/or porosity and/or roughness and which is coated with a bioactive material by either forming the inner bearing surface layer and subsequently applying the backing layer to it, or forming the backing layer and applying the inner bear surface layer to it.

When preforming the bearing surface layer the backing layer can be applied to it by sputtering, plasma spraying and/or vapor deposition. If the backing layer is preformed the inner bearing surface layer can be provided by molding. The backing layer can be arranged to have a porosity which varies from its inner to its outer sides to form an outer porous surface. The bioactive material can be applied by sputtering, plasma spraying or chemical deposition such as chemical vapor deposition. Any well known deposition method can be used.

### BRIEF DESCRIPTION OF THE DRAWINGS

The invention can be performed in many ways and some embodiments will not be described by way of example and with reference to the accompanying drawing in which:

FIG. 1 is a diagrammatic cross-section through an acetabular cup according to the present invention;

FIG. 2 shows an alternative embodiment and method of making a cup according to the invention;

FIG. 3 shows a third alternative embodiment and method of making it;

FIG. 4 shows a fourth alternative embodiment and a method of making the same;

FIG. 5 shows a fifth alternative embodiment and a method of making the same;

FIG. 6 shows a sixth alternative embodiment and a method of making the same;

FIG. 7 shows a seventh alternative embodiment and a method of making the same; and

FIG. 8 shows a eighth alternative embodiment and a method of making the same.

### DETAILED DESCRIPTION

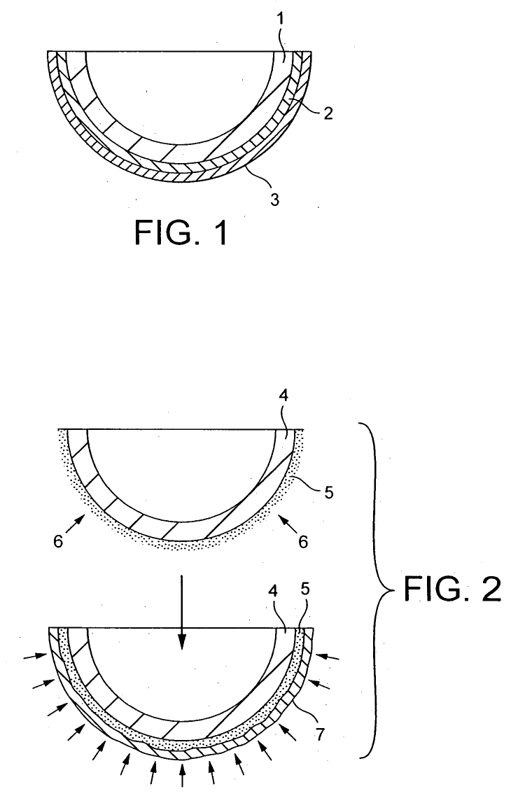

As shown in FIG. 1, a prosthetic acetabular cup, according to the invention, comprises a bearing surface layer 1 which is injection molded of a composite material including PEEK resin and at least 20 to 40% short carbon fibers. The material can be substantially as set out in U.S. Pat. No. 6,638,311 the teachings of which are incorporated herein by reference, see for example pitch based carbon fibers mixed with a PEEK resin (IC Grade 150 g). The material can be palletized and the carbon fibers can be chopped fibers with an average diameter of 8 μm and an average length of 20 μm. The pellets can be molded into an acetabular cup and the fibers loading in the specimens can be arranged to range from 20% to 40%. Again, if desired, the cup can be shaped as set out in EP-A-93 300 413.7 or U.S. Pat. No. 6,638,311.

Commercially pure titanium particles are then sputtered with a plasma torch under vacuum or under gas such as argon to form a backing layer 2. The outer side of the cup can be roughened prior to this metal coating. Hydroxyapatite (HAP) is then sputtered with a plasma torch onto the outer surface of the backing layer, as indicated by reference numeral 3.

The ensuing structure provides a prosthetic acetabular cup which has an inner bearing layer made from the composite material which has a natural elasticity for natural load distribution to the bone and provides a high wear-resistant bearing surface. The backing layer 2 creates a barrier between the composite material and the bone cells and/or provides an appropriately roughness for bone cell attachment and/or provides open porosity for bone cell ingrowth and the bioactive material 3 encourages the bone cells apposition and development. The use of a metallic material for the backing layer 2 provides an opaque marker for X-rays.

FIG. 2 shows a second method and embodiment. In this arrangement the bearing surface layer 4 is made in a similar manner to that described with regard to FIG. 1, that is the composite structure is injection molded. A second layer is then formed by sputtering or spraying commercially pure titanium particles with a plasma torch under vacuum to provide a backing layer 5, the sputtering being indicated by arrows 6. At the beginning of the coating process the size of the titanium particles is small and increases in order to form an interconnected porosity which increases over the width of the structure. The porous structure 5 is then coating with HAP by deposition in order to ensure a continuous HAP layer indicated by reference numeral 7 (Pore size: 400 μm nominal, irregular structure.) The porosity of the layer 5 assists in providing a structure for mechanical fixation after dissolution of the bioactive layer 7.

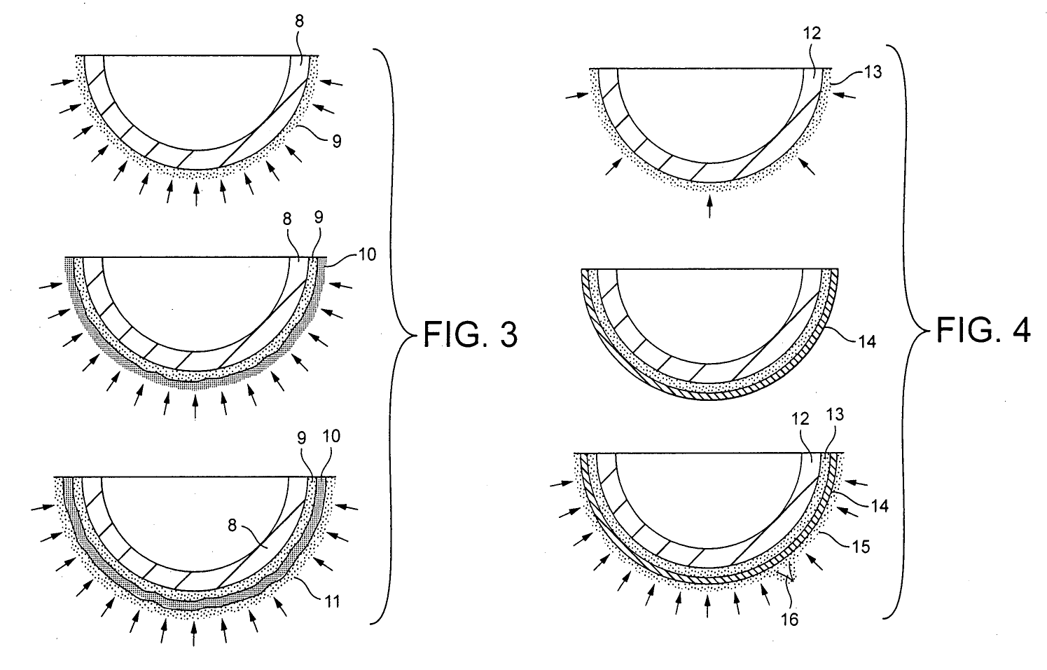

FIG. 3 shows a three stage method in which the inner bearing layer 8 and backing layer 9 are made in a similar manner to that described in the method shown in FIG. 2. A further layer 10 is then formed on the backing layer by plasma spraying or sputtering a mixture of titanium powder and hydroxyapatite particles and finally the second layer 10 is then sputtered with pure hydroxyapatite powder, indicated by reference numeral 11. The hydroxyapatite particles 11 embedded in the titanium layer 10 dissolve and are replaced by bone trabeculae that enhance the mechanical fixation of the implant.

In the description and method shown in FIG. 4 the bearing layer 12 and layer 13 are made in a similar manner described with regard to FIGS. 1 to 3. The size of the pure titanium particles 13 and the surface roughness is smooth enough to tolerate the formation of a titanium structure 14 which is obtained by a laser sintering process, for example using the process described in U.S. patent application Ser. No. 10,704,270 filed on Nov. 7, 2004 entitled Laser-Produced Porous Surface. The resulting porous structure is then coated with HAP by deposition/sputtering in order to ensure a continuous HAP layer 15. The construction creates a modulus gradient from composite to HA and this can provide a better mechanical construction.

A benefit of this construction and method is that it provides a predetermined type of porosity (size, extent), some fixation fixtures can be deposited, as indicated by reference numeral 16, and a porosity, density or a combination can be provided can be provided, for example fins or spikes. If barbs are included they can provide additional multidirectional torsional stability.

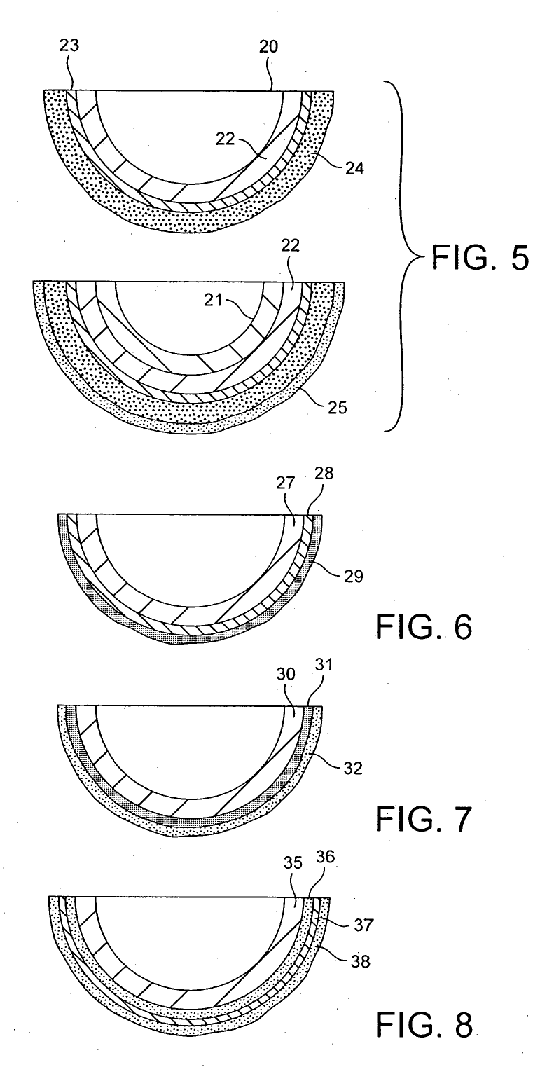

FIG. 5 shows a method and construction which utilizes a preformed metal shell which can be used as an insert. The shell is indicated by reference numeral 20 and has an inner surface of specified structure, roughness, and retentive features to permit engagement of a plastic composite bearing surface, indicated by reference numeral 21.

The metal perform may be made as a graded metal structure by, for example, laser sintering using titanium and having an overall thickness of 2-3 mm. The perform comprises an inner surface layer 22 which is porous to retain the plastic composite bearing surface 21, a dense layer 23 which acts as a barrier layer to stop ingress of the plastic/composite into the metallic structure and an outer layer 24 which is of controlled interconnected porosity and is intended for bone ingrowth. This has a nominal porosity of 400 μm which is able to sustain the bone ingrowth referred to above.

The preformed metal insert is made as shown at the upper part of FIG. 5 and the composite material bearing surface layer 21 is subsequently molded to it. The outer surface of the metallic structure is then coated with HAP, indicated by reference numeral 25, by sputtering or chemical deposition. The particle size of the porous layer 22 can be 1 mm to allow the composite to infiltrate and to be retained.

In the construction shown in FIG. 6 the bearing layer 27 is made in a similar manner to the arrangements shown in FIGS. 1 to 4, for example by injection molding, and is then coated with a thin titanium layer 28 coating using plasma vapor deposition (PVD) in order to form a very thin titanium barrier between the composite material and the bone cells. A layer of hydroxyapatite 29 is then applied to form a continuous layer without damaging the titanium layer previously applied.

In the construction and method shown in FIG. 7 a bearing surface layer 30 is first made from a composite material including PEEK resin and short carbon fibers by injection molding. PEEK particles are then sputtered by a plasma torch to create a barrier backing layer 31 to prevent ingrowth of the bone cells. At the start of the sputtering process the size of the particles is small and increases in order to form a porous structure. The porous structure layer 31 is then coated with hydroxyapatite, as indicated by reference numeral 32, by any process which will provide a continuous layer.

In the embodiment and method shown in FIG. 8 a composite bearing surface layer 35 is formed by a similar process to that used in the previous FIGS. 1 to 4 and 7 by injection molding. PEEK particles are sputtered by a plasma torch to provide a predetermined roughness in a layer indicated by reference numeral 36. A thin titanium layer 37 is now applied by a plasma torch under vacuum to form a barrier between the PEEK material layer 36 and the bone cells and the porous structure is then coated with a layer of hydroxyapatite, indicated by reference numeral 38, by any process that will provide a continuous layer.

In all the above examples the composite material is preferably PEEK reinforced with 30% carbon fibers produced to actual shape by injecting molding. The part can also be formed by a combination of molding/extrusion and machining to final shape.

Any material such as tantalum or niobium could be used as an alternative to titanium. The hydroxyapatite bioactive layer can be enhanced or replaced by a coating with bone morphogenic proteins in any of the examples, or even omitted.

As described above the hydroxyapatite layer can be applied by plasma torch sputtering for non-porous surfaces. Onto porous surfaces, deposition process or any process that will ensure full covering of the open porous surface and allow thickness control can be applied. Such processes can be deposition or laser ablation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of forming a prosthetic bearing element comprising:
injection molding a bearing layer of PEEK resin having 20 to 40% carbon fiber to form an inner bearing surface;
sputtering metal particles on an outer non-bearing surface of said molded bearing to form a porous backing layer; and
sputtering hydroxyapatite onto the metal backing layer to form an outer surface of the prosthetic bearing element.

2. The method as set forth in claim 1 further comprising plasma spraying PEEK on the outer surface of the bearing layer prior to applying the metal layer.

3. The method as set forth in claim 1 wherein the metal particle size is varied to form an interconnected porosity which increases towards the outer surface as the layer is built up.

4. The method as set forth in claim 3 wherein the particle size increases from smaller to larger to form the increasing interconnected porosity.

5. The method as set forth in claim 1 wherein a mixture of metal and hydroxyapatite particles is sputtered onto the backing layer.

6. The method as set forth in claim 1 wherein the bearing layer has about 30% short carbon fibers.

7. The method as set forth in claim 1 further comprising a metal backing between the PEEK bearing and the porous metal layer for blocking tissue ingrowth beyond the porous metal layer.

8. A prosthetic acetabular cup comprising a bearing surface layer made from a composite material which includes PEEK resin and at least 20% to 40% short carbon fibers, and a backing layer or layers to provide a barrier and/or porosity and/or roughness.

9. The prosthetic acetabular cup as set forth in claim 8 in which the backing layer is made from metal.

10. The prosthetic acetabular cup as set forth in claim 9 in which the metal is selected from the group consisting of titanium, tantalum, titanium alloy, cobalt chrome alloy or niobium.

11. The prosthetic acetabular cup as set forth in claim 8 in which the backing layer is made from pure PEEK resin to produce a barrier between the composite material or the surface layer and the bone cells in which it will be used.

12. The prosthetic acetabular cup as set forth in claim 8 in which the backing layer is coated with a bioactive material.

13. The prosthetic acetabular cup as set forth in claim 12 in which the bioactive material is hydroxyapatite (HAP) and/or bone morphogenic proteins (BMP).

14. A method of making an acetabular cup comprising forming a bearing surface layer from a composite material including PEEK resin and at least 20% to 40% short carbon fibers; forming a backing layer or layers over said bearing surface to provide a barrier and/or porosity and/or roughness, and coating the backing layer with a bioactive material.

15. The method as set forth in claim 14 wherein the backing layer is applied after forming the bearing surface layer.

16. The method as set forth in claim 14 wherein the backing layer is formed first and the inner bearing layer is subsequently applied to the backing layer.

17. The method as set forth in claim 14 further comprising preforming the bearing surface layer and applying the backing layer or layers by sputtering and/or chemical deposition.

18. The method as set forth in claim 14 further comprising preforming the backing layer and providing the inner bearing surface layer by molding.

19. The method as set forth in claim 14 further comprising a backing layer the porosity of which varies from its inner to its outer sides to form an outer porous surface.

20. The method as set forth in claim 14 further comprising applying the bioactive material by sputtering or chemical deposition.

21. The prosthetic acetabular cup as set forth in claim 1 wherein the metal is selected from the group consisting of titanium, tantalum, titanium alloy, cobalt chrome alloy or niobium.
